(19) United States
(12) Patent Application Publication
(10) Pub. No.: US 2023/0323390 A1
(43) Pub. Date: Oct. 12, 2023

(54) METHODS AND COMPOSITIONS FOR EXPRESSING PHENYLALANINE HYDROXYLASE

(71) Applicant: Sangamo Therapeutics, Inc., Brisbane, CA (US)

(72) Inventor: Gustavo de Alencastro, Brisbane, CA (US)

(73) Assignee: Sangamo Therapeutics, Inc., Brisbane, CA (US)

(21) Appl. No.: 18/001,342

(22) PCT Filed: Jun. 11, 2021
(86) PCT No.: PCT/US2021/037087

§ 371 Date: Dec. 9, 2022

Related U.S. Application Data
(60) Provisional application No. 63/037,857, filed on Jun. 11, 2020.

(51) Int. Cl. C12N 15/86; C12N 9/02; A61P 3/00

(52) U.S. Cl. CPC C12N 15/86; C12N 9/0071; A61P 3/00; C12Y 114/16001; C12N 2750/14143

(57) ABSTRACT

The present disclosure provides expression constructs comprising a phenylalanine hydroxylase (PAH) transgene and methods of using the constructs for treating phenylketonuria (PKU).

### CROSS-REFERENCE TO RELATED APPLICATION

This application claims priority from U.S. Provisional Patent Application 63/037,857, filed Jun. 11, 2020. The disclosure of that priority application is incorporated by reference herein in its entirety.

### SEQUENCE LISTING

The instant application contains a Sequence Listing which has been submitted electronically in ASCII format and is hereby incorporated by reference in its entirety. The electronic copy of the Sequence Listing, created on Jun. 2, 2021, is named 025297_W0018_SL.txt and is 14,506 bytes in size.

### BACKGROUND OF THE INVENTION

Phenylketonuria (PKU) is an autosomal recessive disorder that results in decreased metabolism of the amino acid phenylalanine (Phe) and consequently high plasma Phe levels. There are several types of PKU: classic PKU, moderate PKU, mild PKU, and variant PKU. Variant PKU, also known as mild hyperphenylalaninemia, “hyperphe” or “mild HPA,” has the least severe defects in phenylalanine metabolism. While healthy individuals have plasma Phe levels of about 30-60 μmol/L, the plasma Phe levels are greater than about 1,200 μmol/L for “classic” PKU patients, are about 900-1,200 μmol/L for “moderate” PKU patients, are about 600-900 μmol/L for “mild” PKU patients, and are about 360-600 μmol/L for “variant” PKU patients. Individuals with plasma Phe levels ranging between about 120-360 μmol/L are considered “benign” or “mild” PKU patients who typically do not require therapeutic interventions such as a Phe-restricted diet. See, e.g., Camp et al., Mol Genet Metabol. (2014) 112:87-122

PKU dramatically affects myelination and white matter tracts in infants. Untreated children often fail to attain early developmental milestones, develop microcephaly, and demonstrate progressive impairment of cerebral function. Hyperactivity, EEG abnormalities, seizures, and severe learning disabilities are major clinical problems later in life. Further, a characteristic “musty or mousy” odor on the skin, as well as a predisposition for eczema, persist throughout life in the absence of treatment. There are about 50,000 PKU patients in the world.

PKU usually results from loss-of-function mutations in the gene for the enzyme phenylalanine hydroxylase (phenylalanine-4-hydroxylase or PAH), a 52 kD protein that together with co-factor tetrahydrobiopterin (BH4), converts Phe to tyrosine. PAH is expressed mainly in the liver and kidney. More than 500 disease-causing mutations have been found in the PAH gene, including R408W, a common mutation among Europeans.

PKU is not currently curable, but treatment options are available. At present, treatment relies on early diagnosis by newborn screening. If PKU is detected, treatment typically involves dietary restrictions, i.e., a lifelong diet low in Phe with supplements including tyrosine, which the body cannot produce without PAH. The restrictive diet severely impacts patients' quality of life. Further, compliance with the restrictive diet is extremely burdensome. Also, the diet may not fully reduce blood Phe levels to the desired range and may not prevent long-term neurocognitive impairment. Supplementation with BH4 (Kuvan®/sapropterin dihydrochloride) has yielded some improvement in Phe levels in about 20-30% of patients, but BH4 still needs to be used in combination with a restrictive diet. Accordingly, there remains a high unmet medical need for improved PKU treatment that provides long-lasting benefits for most patients.

### SUMMARY OF THE INVENTION

The present disclosure provides a nucleic acid construct comprising an expression cassette for human phenylalanine hydroxylase (PAH), wherein the expression cassette comprises a PAH-coding sequence linked operably to a mammalian promoter, the expression cassette further comprising a first RNA transcript enhancing element and a second RNA transcript enhancing element.

In some embodiments, the construct further comprises an enhancer, e.g., the human apolipoprotein E (ApoE) enhancer or an enhancer that comprises SEQ ID NO: 2.

In some embodiments, the construct further comprises a promoter that is active in human liver cells (e.g., hepatocytes), such as a human alpha-1 antitrypsin (AAT) promoter or a promoter that comprises SEQ ID NO: 3.

In some embodiments, the PAH-coding sequence encodes a human PAH protein. In certain embodiments, the human PAH protein comprises SEQ ID NO: 9 or an amino acid sequence that is at least 95% identical to SEQ ID NO: 9. In certain embodiments, the PAH-coding sequence comprises SEQ ID NO: 5 or a codon-optimized variant of SEQ ID NO: 5.

In some embodiments, the first RNA transcript enhancing element comprises an intron (e.g., an HBB-IGG intron or an intron that comprises SEQ ID NO: 4). In some embodiments, the second RNA transcript enhancing element comprises a WPRE element (e.g., a WPRE element mutated from wildtype WPRE or comprising SEQ ID NO: 6). In certain embodiments, the first and second RNA transcript enhancing elements are located upstream and downstream, respectively, of the PAH-coding sequence. For example, the first and second RNA transcript enhancing elements may flank the PAH-coding sequence.

In some embodiments, the expression cassette comprises a polyA signal, such as a bovine growth hormone polyA signal or a polyA signal that comprises SEQ ID NO: 7.

In some embodiments, the expression cassette comprises, from 5′ to 3′, an enhancer, a promoter described herein, a first RNA transcript enhancing element described herein, a PAH-coding sequence described herein, a second RNA transcript enhancing element described herein, and a polyA signal described herein. In certain embodiments, the construct comprises, from 5′ to 3′, SEQ ID NOs: 2, 3, 4, 5, 6, and 7. In certain embodiments, the construct comprises SEQ ID NO: 10.

In some embodiments, the construct is a viral construct. The construct may be, for example, an adeno-associated viral construct. In certain embodiments, the construct comprises AAV inverted terminal repeats (ITRs) (e.g., AAV2 ITRs). In particular embodiments, the construct comprises a 5′ ITR and a 3′ ITR comprising SEQ ID NOs: 1 and 8, respectively.

The present disclosure also provides a recombinant adeno-associated virus (rAAV) comprising a construct as described herein. The rAAV may have a natural or chimeric serotype, be of serotype AAV6, AAV7, AAV8, or AAV9, comprise capsid proteins derived from AAV2, AAV6, AAV7, AAV8, and/or AAV9, comprise hybrid capsid proteins derived from more than one AAV serotype (e.g., from AAV2, AAV6, and AAV9); and/or be hepatotropic. In some embodiments, the rAAV is rAAV6. In some embodiments, the rAAV is rAAV9. In some embodiments, the rAAV is rAAV2/9. The rAAV may be produced in insect cells (e.g., Sf9 cells) or mammalian cells (e.g., HEK293 cells), in certain embodiments.

The present disclosure also provides a cell for producing an rAAV as described herein, wherein the cell comprises a nucleotide sequence coding for capsid proteins VP1, VP2, and VP3 of the rAAV, and a construct as described herein. In some embodiments, the cell is an insect cell (e.g., an Sf9 cell). In some embodiments, the cell is a mammalian cell (e.g., a HEK293 cell).

The present disclosure also provides a method of producing an rAAV as described herein, comprising culturing a cell as described herein under conditions that allow packaging of the rAAV, and isolating the packaged rAAV.

The present disclosure also provides a pharmaceutical composition comprising a construct as described herein or an rAAV as described herein, and a pharmaceutically acceptable excipient.

The present disclosure also provides a method of expressing human phenylalanine hydroxylase (PAH) protein in a mammalian cell, the method comprising introducing to the cell a construct as described herein, an rAAV as described herein, or a composition as described herein. In some embodiments, the mammalian cell is a human cell (e.g., in or from a human in need of reduction of plasma phenylalanine level). In certain embodiments, the mammalian cell is a liver cell (e.g., a hepatocyte). The present disclosure also provides a human cell engineered by a method described herein.

The present disclosure also provides a method of treating PKU in a human patient, the method comprising administering to the patient a therapeutically effective amount of a construct as described herein, an rAAV as described herein, a composition as described herein, or a cell as described herein.

The present disclosure also provides a method of treating PKU in a human patient, the method comprising administering to the patient a therapeutically effective amount of a nucleic acid construct comprising an expression cassette for human phenylalanine hydroxylase (PAH), wherein the construct is delivered by (e.g., packaged in) an rAAV that is rAAV9 or rAAV2/9. In some embodiments, the nucleic acid construct is a construct described herein. In some embodiments, the rAAV9 or rAAV2/9 is produced in insect cells (e.g., Sf9 cells), or in mammalian cells (e.g., HEK293 cells). In some embodiments, the method comprises administering to the patient a therapeutically effective amount of the rAAV9 or rAAV2/9, a pharmaceutical composition comprising the construct and a pharmaceutically acceptable excipient, or a mammalian cell (e.g., a liver cell, such as a hepatocyte) comprising the construct.

In the methods of treatment described herein, the PKU may be, e.g., classic, moderate, mild, or variant PKU. In some embodiments, the rAAV is administered intravenously or via direct injection. In some embodiments, the treatment results in (i) a decrease in phenylalanine (Phe) levels by at least 30% or by at least 90%, and/or (ii) Phe levels of less than 360 μmol/L, in a blood sample from the patient.

The present disclosure also provides the use of a nucleic construct comprising an expression cassette for human PAH (e.g., a construct as described herein), an rAAV as described herein, a composition as described herein, or a cell as described herein, for the manufacture of a medicament for treating phenylketonuria according to a method as described herein.

The present disclosure also provides a nucleic construct comprising an expression cassette for human PAH (e.g., a construct as described herein), an rAAV as described herein, a composition as described herein, or a cell as described herein, for use in treating phenylketonuria according to a method as described herein.

It is understood that the PAH transgenes of the present disclosure, and expression constructs, vectors, and modified cells comprising them as described herein, may be used in a method of treatment as described herein, may be for use in a treatment as described herein, and/or may be for use in the manufacture of a medicament for a treatment as described herein. The present disclosure also provides kits and articles of manufacture comprising the PAH transgenes, expression constructs, vectors, or modified cells described herein.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE DRAWINGS

FIG. 1 is a schematic diagram showing an exemplary PAH cDNA construct (“Vector 1”) comprising an apolipoprotein E (APOE) enhancer linked to a human alpha 1 antitrypsin (hAAT) promoter, a hemoglobin beta (HBB)/immunoglobulin G (IgG) (HBB-IGG) chimeric intron, a wild type human PAH coding sequence (PAHwt), a mutated woodchuck hepatitis virus post-transcriptional regulatory element (WPRE) sequence, and a bovine growth hormone (bGH) polyA signal sequence.

FIG. 2 is a panel of photographs showing Western blot analysis of PAH protein levels after transduction of murine Hepa1-6 liver cells or human primary hepatocytes with viral Vector 1, Vector 3, Vector 5, Vector 6, Vector 8, Vector 15, or Vector 16. Human primary hepatocytes: MOI (left to right) was 600K, 600K, 600K, 200K, 200K, and 200K. Hepa1-6 cells: MOI (left to right) was 500K, 500K, 100K, and 100K. Cells were harvested five days after transduction. The PAH protein was detected using an antibody to human PAH. An antibody to HSP90 was used as a loading control.

FIG. 3 is a panel of graphs showing plasma Phe levels (μg/mL) over time in Enu2 PKU mice treated with Vector 1, Vector 3, Vector 15 and Vector_cDNA4 packaged in an AAV6 viral vector. Females: circles. Males: triangles. “IS”: Immunosuppressant. “Form Buffer”: formulation buffer.

FIG. 4 is a graph showing plasma Phe levels over time in female Enu2 PKU mice treated with Vector 1 packaged in AAV6 or AAV9. Formulation buffer (“Formulation”) is used as a control. The dashed line represents normal plasma Phe levels.

### DETAILED DESCRIPTION OF THE INVENTION

The present disclosure provides methods and compositions for treating phenylketonuria (PKU). The treatment involves introduction to a PKU patient an expression construct encoding a functional PAH (e.g., a wild type PAH or a PAH having at least 90% or more enzymatic activity of a wild type PAH). The functional PAH protein expressed from the expression construct reduces plasma Phe levels in the patient and alleviates or prevents PKU symptoms.

In some embodiments, the expression construct specifically targets hepatocytes in the patient, where the construct may be delivered by a hepatotropic recombinant virus (e.g., AAV6, AAV7, AAV8, or AAV9, or pseudotypes or hybrids derived therefrom). The recombinant virus may transduce at least 1%, 5%, 10%, 15%, 20%, 25%, 30%, 35%, 40%, 45%, 50%, 55%, 60%, 65%, 70%, 75%, or more of the patient's hepatocytes. In some embodiments, at least 10% of the patient's hepatocytes are transduced to express wild-type levels of PAH.

In some embodiments, the present treatment of a PKU patient introduces the PAH enzyme in a targeted tissue (e.g., the liver) in the patient, e.g., by 10-, 20-, 30-, 40-, 50-, 60-, 70-, 80-, 90-, 100-, 500-, or 1000-fold or more.

In some embodiments, the present treatment of a PKU patient results in a decrease in plasma Phe levels in the patient by at least about 20%, 30%, 40%, 50%, 60%, 70%, 75%, 80%, 85%, 90%, or 95%. In certain embodiments, the decrease in plasma Phe levels lasts for at least 30, 60, 90, 100, 110, 120, 130, 140, 150, 160, 170, or 180 days, 4 months, 5 months, 6 months, or 9 months, or 1, 2, 3, 4, 5, 10, 20, 40, or 50 years, or for the lifetime of the patient. The patient may be treated again as needed, for example, when the patient's plasma Phe levels start to rise.

In some embodiments, the patient to be treated has plasma Phe levels greater than about 1,200 μmol/L (classic PKU), between about 900 and about 1,200 μmol/L (moderate PKU), between about 600 and about 900 μmol/L (“mild PKU”), or between about 360 and about 600 μmol/L (variant PKU). In particular embodiments, the patient to be treated has plasma Phe levels greater than about 360, 600, 900, or 1200 μmol/L. In certain embodiments, a patient to be treated has plasma Phe levels greater than 600 μmol/L. In some embodiments, the present treatment results in plasma Phe levels in the patient of less than 1200, 900, 600, 360, or 120 μmol/L. In certain embodiments, the treatment reduces the patient's plasma Phe to levels below 360 μmol/L or within the normal range.

The present treatment methods are particularly effective in treating PKU as compared to standard of care and will substantially increase PKU patients' quality of life. In some embodiments, the treatment may improve an affected child's physical and/or neurological development, as assessed by physical examination and neurocognitive assessments (e.g., as scored by ADHD-RS or PKU-POMs). In some embodiments, there will be a reduced need or no need to place the patient on a restrictive diet or a supplemented diet. The treatment may be repeated only as needed, and may be infrequently, for example, at a frequency of about 1, 2, 5, 10, 20, 30, or more years, such as at a frequency of about 5 or more years, or such as at a frequency of about 10 or more years. In some instances, the patient may be treated concurrently with a less restrictive diet or with BH4 and/or tyrosine supplements.

### I. PAH Expression Constructs

The present disclosure provides a highly efficient expression construct comprising an expression cassette for human PAH. The expression cassette comprises a PAH transgene (a coding sequence without introns (e.g., a cDNA) or with one or more introns) linked operatively to one or more transcriptional regulatory elements. As used herein, “transcriptional regulatory elements” refer to nucleotide sequences in the expression construct that control expression of the PAH coding sequence, for example, by regulating the tissue-specific expression patterns of the PAH coding sequence, the transcription efficiency of the PAH coding sequence, the physical stability of the RNA transcripts, processing of the RNA transcripts, transport (e.g., nuclear export) of the RNA transcript, and/or the translation efficiency of the RNA transcripts. These elements may be one or more of an enhancer, a promoter, an intron, a post-transcriptional regulatory element (e.g., a WPRE sequence), a polyadenylation signal, and any combination thereof. In some embodiments, the expression construct comprises all of said components.

A. PAH Coding Sequence

The human PAH gene is located at chromosome 12q23.2 and has 15 exons (NCBI Database Gene ID 5053). In some embodiments, the PAH transgene in the expression cassette encodes a wild type human PAH having 452 amino acids. An example of wild type human PAH has the following sequence:

MSTAVLENPG LGRKLSDFGQ ETSYIEDNCN QNGAISLIFS
LKEEVGALAK VLRLFEENDV NLTHIESRPS RLKKDEYEFF
THLDKRSLPA LTNIIKILRH DIGATVHELS RDKKKDTVPW
FPRTIQELDR FANQILSYGA ELDADHPGFK DPVYRARRKQ
FADIAYNYRH GQPIPRVEYM EEEKKTWGTV FKTLKSLYKT
HACYEYNHIF PLLEKYCGFH EDNIPQLEDV SQFLQTCTGF
RLRPVAGLLS SRDFLGGLAF RVFHCTQYIR HGSKPMYTPE
PDICHELLGH VPLFSDRSFA QFSQEIGLAS LGAPDEYIEK
LATIYWFTVE FGLCKQGDSI KAYGAGLLSS FGELQYCLSE
KPKLLPLELE KTAIQNYTVT EFQPLYYVAE SENDAKEKVR
NFAATIPRPF SVRYDPYTQR IEVLDNTQQL KILADSINSE
IGILCSALQK IK (SEQ ID NO: 9; UniProtKB/
Swiss-Prot : P00439)

In some embodiments, the PAH coding sequence encodes a functionally active isoform or variant of the above sequence. The encoded polypeptide may share at least 70%, 80%, 85%, 90%, or 95% (e.g., at least 96%, 97%, 98%, or 99%) sequence identity with SEQ ID NO: 9. By way of example, the PAH coding sequence may encode a PAH protein having functionally silent amino acid changes at sites known to be priming epitopes for endogenous immune responses, a PAH having deletions, resulting in a less immunogenic protein.

In some embodiments, the PAH coding sequence comprises a wild type human PAH cDNA. An example of such a cDNA has the following sequence:

ATGTCCACTGCGGTCCTGGAAAACCCAGGCTTGGGCAGGAAACTCTCTGA
CTTTGGACAGGAAACAAGCTATATTGAAGACAACTGCAATCAAAATGGTG
CCATATCACTGATCTTCTCACTCAAAGAAGAAGTTGGTGCATTGGCCAAA
GTATTGCGCTTATTTGAGGAGAATGATGTAAACCTGACCCACATTGAATC
TAGACCTTCTCGTTTAAAGAAAGATGAGTATGAATTTTTCACCCATTTGG
ATAAACGTAGCCTGCCTGCTCTGACAAACATCATCAAGATCTTGAGGCAT
GACATTGGTGCCACTGTCCATGAGCTTTCACGAGATAAGAAGAAAGACAC
AGTGCCCTGGTTCCCAAGAACCATTCAAGAGCTGGACAGATTTGCCAATC
AGATTCTCAGCTATGGAGCGGAACTGGATGCTGACCACCCTGGTTTTAAA
GATCCTGTGTACCGTGCAAGACGGAAGCAGTTTGCTGACATTGCCTACAA
CTACCGCCATGGGCAGCCCATCCCTCGAGTGGAATACATGGAGGAAGAAA
AGAAAACATGGGGCACAGTGTTCAAGACTCTGAAGTCCTTGTATAAAACC
CATGCTTGCTATGAGTACAATCACATTTTTCCACTTCTTGAAAAGTACTG
TGGCTTCCATGAAGATAACATTCCCCAGCTGGAAGACGTTTCTCAGTTCC
TGCAGACTTGCACTGGTTTCCGCCTCCGACCTGTGGCTGGCCTGCTTTCC
TCTCGGGATTTCTTGGGTGGCCTGGCCTTCCGAGTCTTCCACTGCACACA
GTACATCAGACATGGATCCAAGCCCATGTATACCCCCGAACCTGACATCT
GCCATGAGCTGTTGGGACATGTGCCCTTGTTTTCAGATCGCAGCTTTGCC
CAGTTTTCCCAGGAAATTGGCCTTGCCTCTCTGGGTGCACCTGATGAATA
CATTGAAAAGCTCGCCACAATTTACTGGTTTACTGTGGAGTTTGGGCTCT
GCAAACAAGGAGACTCCATAAAGGCATATGGTGCTGGGCTCCTGTCATCC
TTTGGTGAATTACAGTACTGCTTATCAGAGAAGCCAAAGCTTCTCCCCCT
GGAGCTGGAGAAGACAGCCATCCAAAATTACACTGTCACGGAGTTCCAGC
CCCTCTATTACGTGGCAGAGAGTTTTAATGATGCCAAGGAGAAAGTAAGG
AACTTTGCTGCCACAATACCTCGGCCCTTCTCAGTTCGCTACGACCCATA
CACCCAAAGGATTGAGGTCTTGGACAATACCCAGCAGCTTAAGATTTTGG
CTGATTCCATTAACAGTGAAATTGGAATCCTTTGCAGTGCCCTCCAGAAA
ATAAAGTAA (SEQ ID NO: 5; NCBI CCDS9092.1)

In some embodiments, the PAH coding sequence is a degenerate variant (e.g., a codon-optimized variant) of the above cDNA sequence. The cDNA sequence may share at least 70%, 80%, 85%, 90%, or 95% (e.g., at least 96%, 97%, 98%, or 99%) sequence identity with SEQ ID NO: 5. For example, the transgene may be modified from the wild type sequence to provide enhanced biological activity (e.g., to improve expression characteristics) and/or to reduce CpG islands.

B. Transcriptional Regulatory Elements

The expression cassette in the expression construct may contain one or more transcriptional regulatory elements to regulate the expression of the PAH coding sequence in the desired cells (e.g., human hepatocytes and renal cells).

1. Promoter

In some embodiments, the expression cassette contains a mammalian promoter that is constitutively active or inducible in the target cells. Constitutively active promoters include, without limitation, a Rous sarcoma virus (RSV) long terminal repeat (LTR) promoter (optionally with an RSV enhancer), a cytomegalovirus (CMV) promoter (optionally with a CMV enhancer), a CMV immediate early promoter, a simian virus 40 (SV40) promoter, a dihydrofolate reductase (DHFR) promoter, a β-actin promoter, a phosphoglycerate kinase (PGK) promoter, an EFlα promoter, a Moloney murine leukemia virus (MoMLV) LTR, a creatine kinase-based (CK6) promoter, a transthyretin promoter (TTR), a thymidine kinase (TK) promoter, a tetracycline responsive promoter (TRE), a hepatitis B Virus (HBV) promoter, a human α1-antitrypsin (hAAT) promoter, chimeric liver-specific promoters (LSPs), an E2 factor (E2F) promoter, the human telomerase reverse transcriptase (hTERT) promoter, a CMV enhancer/chicken β-actin/rabbit β-globin promoter (CAG promoter; Niwa et al., Gene (1991) 108(2):193-9), an RU-486-responsive promoter, and an albumin promoter (optionally with an enhancer).

In some embodiments, the promoter is a promoter derived from a mammalian (e.g., human) alpha-1 anti-trypsin (AAT) or transthyretin (TTR) gene. An example of a human AAT promoter is shown as SEQ ID NO: 3 (Table 1 below). A variant of this promoter, e.g., a truncated version thereof that still retains the transcriptional regulatory activity of the original promoter, may also be used.

2. Enhancer

In some embodiments, the present expression cassette also contains a transcription enhancer. For example, the enhancer may be or be derived from a mammalian gene (e.g., human apolipoprotein E or ApoE gene), a cis-regulatory module (CRM), a non-naturally occurring Serpin 1 enhancer, or an albumin enhancer. The enhancer may be placed upstream or downstream of the promoter. In certain embodiments, the expression construct comprises more than one copy of an enhancer (e.g., two, three, or four copies of an ApoE enhancer; see, e.g., Okuyama et al., Hum Gen Ther. (1996) 7(5):637-45).

In particular embodiments, an ApoE enhancer is used in conjunction with a human AAT promoter (Miao et al., Mol Ther. (2000) 1(6): 522-32). The promoter/enhancer combination is specifically and highly active in hepatocytes, thereby reducing or preventing expression of the exogenously introduced PAH in non-target tissues.

In certain embodiments, the enhancer comprises SEQ ID NO: 2 (Table 1) or a functional variant thereof

3. Intron

In some embodiments, the expression cassette may contain an intron, placed between the promoter and the PAH coding sequence or downstream of the PAH coding sequence. The intron may increase transgene expression by helping to stabilize mRNA or the activity of enhancers/promoters. The intron may be a natural intron from the PAH gene, or an intron from another gene, an intron derived from more than one gene, or an artificial intron. The intron may be, for example, derived from an intron of a minute virus of mice (MVM) or a mammalian (e.g., human) small bristles (SBR) gene, or a synthetic intron.

In certain embodiments, the intron is an HBB-IgG chimeric intron. The HBB-IgG chimeric intron may comprise a 5′-donor site from the first intron of the human β-globin gene and the branch and 3′-acceptor site from the intron between the leader and the body of an immunoglobulin gene heavy chain variable region (Bothwell et al., Cell (1981) 24:625-37). In particular embodiments, the intron comprises SEQ ID NO: 4 (Table 1), or a functional variant thereof (e.g., a variant that is at least 50%, 60%, 70%, 80% or 90%), which in some embodiments has no open reading frame that is in frame with the PAH coding sequence.

4. Other Elements

The present expression cassette may contain other transcriptional regulatory elements such as elements that regulate or stabilize RNA transcripts. As used herein, transcriptional regulatory elements include those that regulate or stabilize RNA transcripts after transcription has taken place (i.e., post-transcriptional regulatory elements). For example, certain transcriptional regulatory elements may be RNA transcript enhancing elements that may act after transcription has taken place and help increase protein levels by physically stabilizing the RNA transcript, or by facilitating the processing, transportation (e.g., nuclear export), and/or translation efficiency of the transcript.

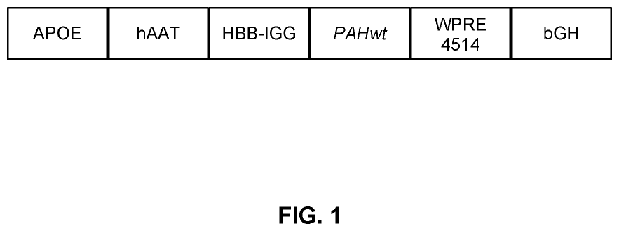

In some embodiments, the construct contains (e.g., downstream or upstream of the PAH coding sequence) a woodchuck hepatitis virus post-transcriptional regulatory element (WPRE). The WPRE may be a wild type sequence or a functional variant thereof. For example, in some embodiments, the WPRE is a mutated WPRE sequence comprising the WPRE mut6 mutations described in Zanta-Boussif et al., Gene Therapy (2009) 16:605-19 and U.S. Pat. No. 10,179,918. The WPREmut6 sequence (SEQ ID NO: 6, Table 1) contains six point mutations (shown in boxes in Table 1) that remove a putative promoter sequence and an ATG start codon in the wild type WPRE sequence. In some embodiments, the mut6 mutations are made in the J04514 WPRE, while in other embodiments, they are made in the J02442.1 WPRE (Ong et al., bioRxiv (2017) doi.org/10.1101/126904). In some embodiments, both an intron and a WPRE are used in one construct to increase mRNA stability, for example, as in Vector 1 shown in FIG. 1.

The expression construct may further comprise a polyA signal downstream of the PAH coding sequence. In some embodiments, the polyA signal is derived from a mammalian gene, such as a bovine growth hormone (bGH) gene, or is a synthetic polyA signal such as the SPA51 sequence. In particular embodiments, the polyA signal is a bGH polyA signal comprising SEQ ID NO: 7 or a functional variant thereof.

In particular embodiments, the expression construct of the present disclosure comprises (e.g., from 5′ to 3′): an ApoE enhancer (e.g., SEQ ID NO: 2), a human AAT promoter (e.g., SEQ ID NO: 3), an HBB-IgG chimeric intron (e.g., SEQ ID NO: 4), a PAH coding sequence (e.g., SEQ ID NO: 5), a WPRE (e.g., SEQ ID NO: 6), and a bGH polyA signal (e.g., SEQ ID NO: 7). See also FIG. 1.

In some embodiments, the expression construct comprises 5′ and 3′ inverted terminal repeat (ITR) sequences, for example, SEQ ID NOs: 1 and 8. In other embodiments, the expression construct further comprises 5′ and 3′ untranslated regions (UTRs), typically comprised of 1-1000 nucleotides, to serve as spacers. UTR spacers may be located, for example, adjacent to ITRs and/or other regulatory elements of the vector. For example, UTRs may be located between sequences corresponding to SEQ ID NOs: 1 and 2 and/or SEQ ID NOs: 7 and 8 of the vector. In particular embodiments, the expression construct comprises SEQ ID NO: 10.

In certain embodiments, the expression cassette also comprises additional regulatory sequences, for example: a Kozak sequence; an internal ribosome entry site; a sequence encoding a self-cleaving peptide (e.g., a 2A peptide) or a furin cleavage site, to allow co-expression of another polypeptide in additional to PAH; or an insulator.

### II. Delivery of PAH Expression Constructs

The expression constructs of the present disclosure may be delivered to target cells by suitable means such as localized injection, systemic injection, electroporation, sonoporation, viral transduction, lipofection, microinjection, biolistics, virosomes, liposomes, immunoliposomes, polycation or lipid:nucleic acid conjugates, and nanoparticles (e.g., lipid nanoparticles). For treating PKU, the expression constructs may be delivered in vivo or ex vivo.

In some embodiments, the expression constructs may be viral vectors and may be delivered to patients through recombinant viruses containing the constructs. The viral vectors contain the PAH expression cassette and minimal viral sequences required for packaging and stable expression with or without integration into a host (if applicable). The missing viral functions are supplied in trans by the packaging cell line used to package the recombinant virus as well as other helper plasmids. The viral vector may be selected from, for example, vaccinia vectors, adenoviral vectors, lentiviral vectors, poxyviral vectors, herpes simplex viral vectors, adeno-associated viral (AAV) vectors, retroviral vectors, and hybrid viral vectors. In part depending on virus type, the PAH expression cassette may be stably integrated into the genome of the target cells, or remain in the cells episomally. Integration into the host genome is possible with retrovirus and lentivirus, and less frequently with AAV. In some embodiments, the recombinant virus may be modified such that it becomes highly specific for the target cells (e.g., hepatocytes). In some embodiments, the recombinant virus (e.g., rAAV) may be replication-defective.

In some embodiments, the expression construct is delivered to the patient as an AAV vector. The AAV vector may be a recombinant AAV (rAAV) containing the expression construct. The AAV vector may retain only the two inverted terminal repeats (ITR) of an AAV (e.g., AAV2), with the PAH expression cassette placed between the two ITRs. AAV vectors are especially suitable for therapeutic gene delivery because they infect both dividing and non-dividing cells, can exist as stable episomal structures for long term expression of the transgene, and have very low immunogenicity. In some embodiments, the transgene may integrate into the patient's genome. In certain embodiments, expression of the AAV-integrated transgene may persist for more than 1, 2, 3, 4, 5, 10, 20, 40, or 50 years following therapeutic treatment, or the transgene may persist for the lifetime of the patient. Any suitable AAV serotype may be used. For example, the AAV may be AAV1, AAV2, AAV3, AAV3b, AAV4, AAV5, AAV6, AAV7, AAV8, AAV8.2, AAV9, AAVrh10, or of a pseudotype (an AAV whose genome such as the ITRs is derived from one serotype such as AAV2 while the capsids are derived from another serotype; e.g., AAV2/8, AAV2/5, AAV2/6, AAV2/9, or AAV2/6/9). See, e.g., U.S. Pat. Nos. 7,198,951 and 9,585,971.

In some embodiments, chimeric AAV is used where the capsid proteins of the AAV are derived from more than one serotype and the AAV generated using a mammalian or insect cell system. For example, the rAAV is produced in insect cells containing baculoviral helper constructs carrying the sequences of an AAV cap gene derived from one or multiple AAV serotypes. In particular embodiments, the chimeric cap gene encodes capsid VP1 and VP2 proteins of AAV1, AAV4, AAV6, AAV7, AAV8, or AAV11 wherein two or more point mutations are introduced to the VP1 and VP2 proteins to remove the sites susceptible to proteolysis in insect cells. The introduced point mutations may be residues identical to those at the corresponding positions in AAV2, AAV3, AAV5, AAV9, or AAV10. In further embodiments, the chimeric cap gene encodes a VP1 protein from AAV6 or AAV9 wherein two or more point mutations are introduced to the phospholipase A2 domain (PLA2) domain of the VP1 protein such that the PLA2 domain is the same as that of AAV2 VP1. In certain embodiments, the AAV cap gene on the helper constructs is derived from a mutated AAV6 cap gene wherein mutations are made to introduce (i) residues from AAV2 such that the VP1 capsid protein expressed from the chimeric cap gene has the same PLA2 domain as AAV2 VP1, and (ii) residues from AAV9 such that the VP1 and VP2 capsid proteins expressed from the chimeric cap gene have increased resistance to proteolysis in insect cells. See also PCT/US2020/018206, the disclosure of which is incorporated by reference herein in its entirety.

In some embodiments, the PAH expression construct is an AAV viral vector and is introduced to the target human cell by a recombinant AAV virion whose genome comprises the construct, including having the AAV Inverted Terminal Repeat (ITR) sequences on both ends to allow the production of the AAV virion in a production system such as an insect cell/baculovirus production system or a mammalian cell production system. The AAV may be engineered such that its capsid proteins have reduced immunogenicity or enhanced transduction ability in humans. In some embodiments, AAV6 or a derivative thereof is used. In some embodiments, AAV9 or a derivative thereof is used.

The recombinant AAVs described herein may be produced using methods known in the art. Any suitable permissive or packaging cell type may be employed to produce the viral particles. For example, mammalian (e.g., 293) or insect (e.g., Sf9) cells may be used as the packaging cell line. The packaging cell line may contain helper plasmid(s) encoding the other AAV genes, namely rep and cap, but lacking ITR sequences. A packing cell line also may be infected with a helper virus such as adenovirus.

In some embodiments, the viral vector is an adenoviral vector. Replication-deficient recombinant adenoviral vectors (Ad) can be produced at high titer and readily infect a number of different cell types. Most Ad vectors are engineered such that a transgene expression cassette replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney, and muscle. Conventional Ad vectors have a large carrying/packaging capacity.

In some embodiments, the viral vector is a retroviral vector. The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats (LTR) with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof. The use of non-integrating lentivirus vectors (IDLV) is also contemplated. ψ2 or PA317 cells may be used to package recombinant retroviruses.

In some embodiments, the PAH expression constructs of the present disclosure may be introduced into the genome of target cells, e.g., by random integration or site-specific integration. Site-specific integration may occur through homologous recombination with or without nuclease-mediated single- or double-stranded DNA breaks (e.g., ZFN, TALEN, CRISPR/Cas9, CRISPR/cpf1, or another nuclease). Non-homologous end joining may also be employed to target the expression construct to the host genome, with or without nuclease-mediated single- or double-stranded DNA breaks. Suitable sites for site-specific integration are, for example, genomic safe harbors, i.e., sites in the genome that are able to accommodate the integration of exogenous sequences in a way that ensures that the inserted sequences can function as expected without causing alterations to the target cell genome that would pose a risk to the patient (Papapetrou and Schambach, Molecular Therapy (2016) 24(4):678-84). Suitable genomic safe harbors for PAH expression include, without limitation, the AAVS1, HPRT, ALB and CCR5 genes. In certain embodiments, the albumin gene serves as the safe harbor locus.

### III. Pharmaceutical Applications

The expression constructs, recombinant viruses, and methods herein may be used to treat human patients (adult patients, juvenile patients, or pediatric patients such as infants, children, and adolescents) suffering from or at risk of developing PKU. The term “treating” encompasses alleviation of symptoms, prevention of onset of symptoms, slowing of disease progression, improvement of quality of life, and/or increased survival.

The present disclosure provides pharmaceutical compositions comprising the expression constructs or recombinant viruses described herein. In some embodiments, the pharmaceutical compositions additionally comprise a pharmaceutically acceptable excipient. Such excipients are determined in part by the particular composition being administered, as well as by the particular method used to administer the composition, and may include, e.g., water, saline (e.g., phosphate-buffered saline), dextrose, glycerol, sucrose, lactose, gelatin, dextran, albumin, or pectin. In addition, the composition may contain auxiliary substances, such as wetting or emulsifying agents, pH-buffering agents, stabilizing agents, or other reagents that enhance the effectiveness of the pharmaceutical composition. The pharmaceutical composition may contain delivery vehicles such as liposomes, nanocapsules, microparticles, microspheres, lipid particles, and vesicles.

Expression constructs or recombinant viruses can be delivered in vivo by administration to a patient, typically by systemic administration (e.g., intravenous, intraperitoneal, subcutaneous, intramuscular, or subdermal injection or infusion). The constructs or viruses may also be delivered locally to the target tissue, e.g., through portal vein delivery to the liver. Alternatively, constructs or viruses can be delivered to cells ex vivo and implanted back to the patient.

The expression constructs, viruses, or engineered cells may be administered in a therapeutically effective amount to treat a PKU condition, i.e., at dosages and for periods of time necessary to achieve a desired result (e.g., a target plasma Phe level or alleviation of a PKU symptom). A therapeutically effective amount may vary according to factors such as the severity of the PKU condition and the age, sex and weight of the patient as well as the underlying PAH mutations.

In some embodiments, a viral vector (e.g., an AAV) comprising an expression construct described herein (e.g., Vector 1) is delivered to the patient at a dose of 1×10¹², 1×10¹³, 1×10¹⁴, or 1×10¹⁵ vg/kg or less. Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the patients/subjects to be treated; each unit containing a predetermined quantity of active compound calculated to produce the desired therapeutic effect in association with the required pharmaceutical carrier. The specification for the dosage unit forms of the present disclosure are generally dictated by and directly dependent on (a) the unique characteristics of the therapeutic agent and the particular therapeutic or prophylactic effect to be achieved, and (b) the limitations inherent in the art of compounding such an active compound for the treatment of sensitivity in individuals.

The present disclosure also provides articles of manufacture, e.g., kits, comprising one or more containers (e.g., single-use or multi-use containers) containing a pharmaceutical composition comprising the expression constructs or recombinant viruses of the present disclosure; optionally an additional biologically active molecule (e.g., another therapeutic agent); and instructions for use. In some embodiments, an article of manufacture of the present disclosure may comprise Vector 1 packaged into AAV6, AAV9, or any chimeric or natural serotypes or variations of AAV6 and AAV9. In some embodiments, the articles of manufacture such as kits include a medical device for administering the expression construct or recombinant virus and/or the other biologically active molecule (e.g., a syringe and a needle); and/or an appropriate diluent (e.g., sterile water and normal saline). The present disclosure also includes methods for manufacturing said articles.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

In order that the present disclosure may be better understood, the following examples are set forth. These examples are for purposes of illustration only and are not to be construed as limiting the scope of the present disclosure in any manner.

### EXAMPLES

### Example 1: Expression of PAH in Hepatic Cells with Vector 1

In this study, recombinant AAV2/6 viruses (i.e., viruses containing the genome of serotype 2 packaged in the capsid from serotype 6) comprising eight different human PAH expression constructs were used to transduce mouse Hepa1-6 cells and human primary hepatocytes. Vector 3 and Vector 15 contain the human PAH cDNA under the control of a liver-specific regulatory promoter and enhancer, and a synthetic polyA sequence, inserted in a self-complementary AAV vector. Both vectors contain liver-specific regulatory elements distinct from the regulatory elements in Vector 1 (FIG. 1). Vector 15 differs from Vector 3 in that it contains a short WPRE3 sequence between the human PAH cDNA and the synthetic polyA sequence (Choi et al., Mol Brain (2014) 7:17). Vectors 5, 6 and 8 contain the same regulatory elements as Vector 1 but with different codon-optimized human PAH cDNAs. “Vector_cDNA4” contains the same regulatory elements as Vector 1 but without the WPRE sequence. Vector 16 contains the same regulatory elements as Vector 1 but with a short WPRE sequence (WPRE3) instead of the WPRE4514 sequence.

Human primary hepatocytes (from Lonza lot no. HUM180871) were cultured in HCM Hepatocyte culture medium (cat no. CC-3198). Hepa 1-6 cells were cultured in DMEM supplemented with penicillin, L-glutamine and 10% fetal bovine serum. For human primary hepatocytes, on Day 0, cells were thawed, counted, and plated at 1.75×10⁵ cells in 0.3 mL medium per well into 48 well plates. On Day 1, all the tested AAV vectors (AAV2/6) were mixed at the appropriate MOI (600K or 200K) with the medium and added to the cells. Cells were incubated at 37° C. with 5% CO₂. On Day 6, cells were harvested, and PAH levels were assessed by Western blot analysis using a human PAH antibody.

For Hepa 1-6, on Day 0, cells were trypsinized, counted, and plated at 1.0×10⁵ cells in 0.5 mL medium per well into 24 well plates. On Day 1, all the tested AAV vectors (AAV2/6) were mixed at the appropriate MOI (500K or 100K) with the medium and added to the cells. Cells were incubated at 37° C. with 5% CO₂. On Day 5, supernatants were collected and cells harvested, and PAH levels were assessed by Western blot analysis using a human PAH antibody.

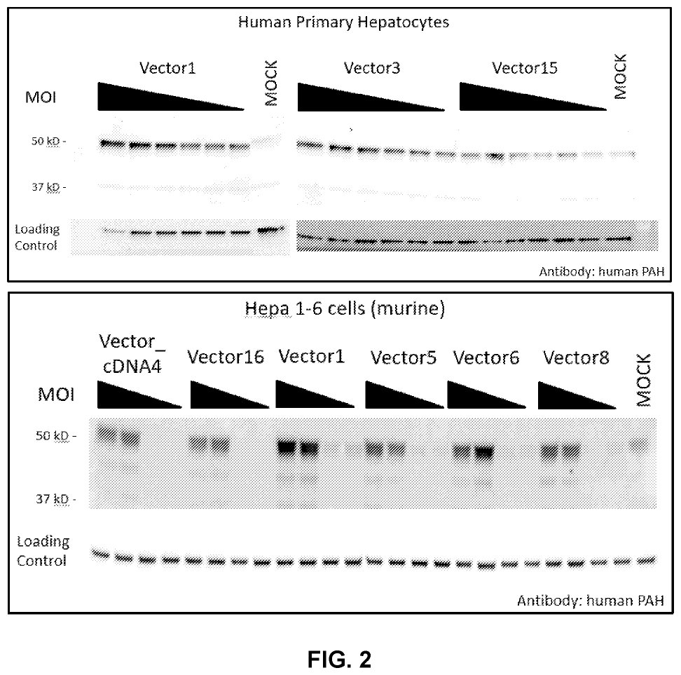

The data show that PAH was expressed from all six constructs, but at the highest level from Vector 1 (FIG. 2). The human PAH protein produced after AAV transduction was shown to be enzymatically functional by quantifying the reduction of Phe in the cell culture media as well as the increase in the Tyr levels, using Phenylalanine Assay Kit (Sigma-Aldrich (cat. no. MAK005)) and Tyrosine Colorimetric Assay Kit (Sigma-Aldrich (cat. no. MAK219)), respectively (data not shown).

### Example 2: Vector 1 Reduces Phenylalanine Levels in a PKU Mouse Model

In order to evaluate the findings from our in vitro analysis (i.e., Vector 1 showing the best potency in expressing human PAH protein among all the tested vectors), in vivo experiments were performed using four different vectors from Example 1 (Vector 1, Vector 3, Vector 15 and Vector_cDNA4) in an animal model of PKU (pah^{Enu2}). Briefly, 3-5 female and male Pah^{Enu2} mice were given a single dose of each AAV vector produced from HEK293 cells. In this study, the Pah^{Enu2} mice were 8-12 weeks old at study initiation. On Day 1, the animals received 1×10¹³ vg/kg through tail vein injection. The volume of injected vector was adjusted based on the animal's weight (10 μL/g). The formulation buffer (PBS, 35 nM NaCl, 1% sucrose, 0.05% pluronic F-68, pH 7.1) containing no viral particles was injected into two males and two females as a negative control. The animals also received 50 mg/kg cyclophosphamide every two weeks, starting on the day prior to AAV injection (with the exception of one group with only males injected with Vector 1 alone). The mice were monitored for three months, with plasma samples collected one week prior to dosing and on Days 14, 28, 56, and 91. Plasma Phe levels were quantified using mass spectrometry.

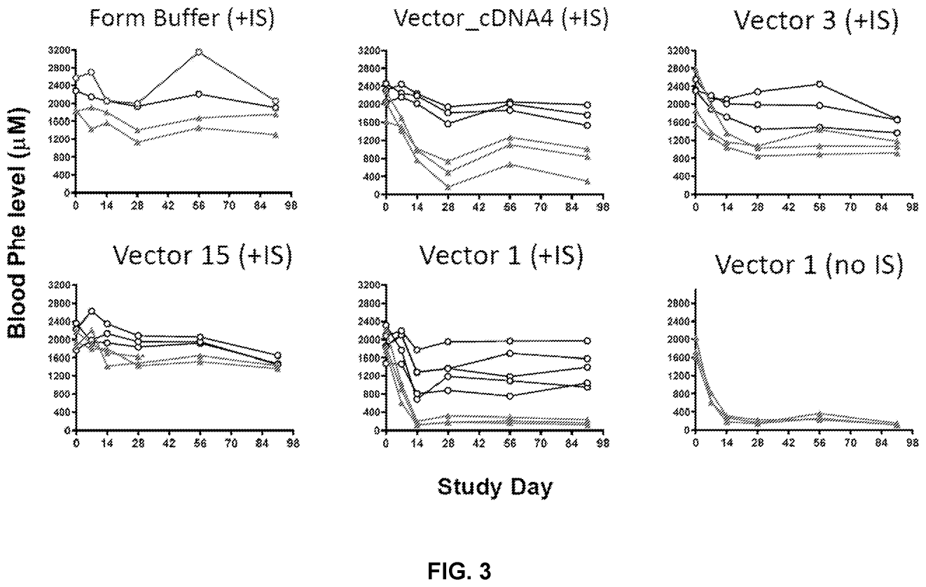

The data of pharmacokinetic evaluation (plasma Phe levels) are presented in FIG. 3. In the figure, each data point represents mean plasma Phe level at the indicated time point. These data show that plasma Phe levels were reduced about 90% in male mice and up to 50% in female mice treated with Vector 1. Wild type animals had a serum Phe level of 51-84 μmol/L. Thus, the treatment achieved close to normal plasma Phe levels during the three month study in male mice.

### Example 3: Comparison of AAV6 and AAV9 Delivery of Vector 1 in PKU Female Mice without Immunosuppressant Treatment

To compare the efficacy of recombinant AAV6 and AAV9 in delivering Vector 1 in an animal model of PKU, female pah^{Enu2} mice were given both Vector 1 AAV2/6 and Vector 1 AAV2/9 produced in HEK293 cells. In this study, the Pah^{Enu2} mice were 8-12 weeks old at study initiation. On Day 1, the animals (n=4) received Vector 1 AAV2/6 or AAV2/9 at 1×10¹³ vg/kg, or formulation buffer (PBS, 35 nM NaCl, 1% sucrose, 0.05% pluronic F-68, pH 7.1) containing no viral particles, as a single tail vein injection. The volume of injected vector was adjusted based on the animal weight (10 μL/g). The mice were monitored for five months, during which Phe levels in plasma samples were quantified using mass spectrometry.

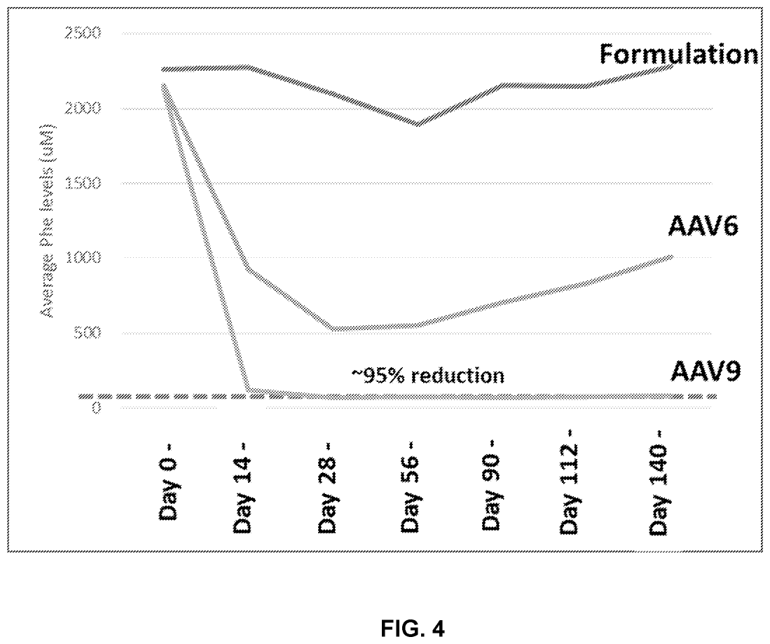

The data of pharmacokinetic evaluation (plasma Phe levels) are presented in FIG. 4. These data show that both Vector 1 AAV2/6 and Vector 1 AAV2/9 reduced plasma Phe levels in the mice, while the formulation buffer control did not alter plasma Phe levels over the same period of time. The data also show that Vector 1 AAV2/9 normalized plasma Phe levels more efficiently than Vector 1 AAV2/6, with about a 95% reduction in plasma Phe levels for at least 140 days. The results support that Vector 1 is highly effective in reducing Phe levels in a PKU mouse model even at the AAV dose used in this experiment.

### SEQUENCES

Table 1 below shows the sequences of an exemplary human PAH expression cassette. The location of each element in the cassette is annotated with the start and end positions of the element in the construct as a whole. SEQ denotes SEQ ID NO.

TABLE 1
Sequence Information

| Element | Location | SEQ | Sequence |
|---|---|---|---|
| 5' ITR | 1-130 | 1 | CTGCGCGCTCGCTCGCTCACTGAGGCCGCCCGGGCAAAGCCCG |
| | | | GGCGTCGGGCGACCTTTGGTCGCCCGGCCTCAGTGAGCGAGCG |
| | | | AGCGCGCAGAGAGGGAGTGGCCAACTCCATCACTAGGGGTTCC |
| | | | T |
| ApoE | 141-461 | 2 | AGGCTCAGAGGCACACAGGAGTTTCTGGGCTCACCCTGCCCCC |
| enhancer | | | TTCCAACCCCTCAGTTCCCATCCTCCAGCAGCTGTTTGTGTGC |
| | | | TGCCTCTGAAGTCCACACTGAACAAACTTCAGCCTACTCATGT |
| | | | CCCTAAAATGGGCAAACATTGCAAGCAGCAAACAGCAAACACA |
| | | | CAGCCCTCCCTGCCTGCTGACCTTGGAGCTGGGGCAGAGGTCA |
| | | | GAGACCTCTCTGGGCCCATGCCACCTCCAACATCCACTCGACC |
| | | | CCTTGGAATTTCGGTGGAGAGGAGCAGAGGTTGTCCTGGCGTG |
| | | | GTTTAGGTAGTGTGAGAGGG |
| Human AAT | 471-863 | 3 | GATCTTGCTACCAGTGGAACAGCCACTAAGGATTCTGCAGTGA |
| promoter | | | GAGCAGAGGGCCAGCTAAGTGGTACTCTCCCAGAGACTGTCTG |
| | | | ACTCACGCCACCCCCTCCACCTTGGACACAGGACGCTGTGGTT |
| | | | TCTGAGCCAGGTACAATGACTCCTTTCGGTAAGTGCAGTGGAA |
| | | | GCTGTACACTGCCCAGGCAAAGCGTCCGGGCAGCGTAGGCGGG |
| | | | CGACTCAGATCCCAGCCAGTGGACTTAGCCCCTGTTTGCTCCT |
| | | | CCGATAACTGGGGTGACCTTGGTTAATATTCACCAGCAGCCTC |
| | | | CCCCGTTGCCCCTCTGGATCCACTGCTTAAATACGGACGAGGA |
| | | | CAGGGCCCTGTCTCCTCAGCTTCAGGCACCACCACTGACCTGG |
| | | | GACAGT |
| HBB-IgG | 867-999 | 4 | GTAAGTATCAAGGTTACAAGACAGGTTTAAGGAGACCAATAGA |
| chimeric | | | AACTGGGCTTGTCGAGACAGAGAAGACTCTTGCGTTTCTGATA |
| intron | | | GGCACCTATTGGTCTTACTGACATCCACTTTGCCTTTCTCTCC |
| | | | ACAG |
| Human PAH | 1052-2410 | 5 | ATGTCCACTGCGGTCCTGGAAAACCCAGGCTTGGGCAGGAAAC |
| CDNA | | | TCTCTGACTTTGGACAGGAAACAAGCTATATTGAAGACAACTG |
| | | | CAATCAAAATGGTGCCATATCACTGATCTTCTCACTCAAAGAA |
| | | | GAAGTTGGTGCATTGGCCAAAGTATTGCGCTTATTTGAGGAGA |
| | | | ATGATGTAAACCTGACCCACATTGAATCTAGACCTTCTCGTTT |
| | | | AAAGAAAGATGAGTATGAATTTTTCACCCATTTGGATAAACGT |
| | | | AGCCTGCCTGCTCTGACAAACATCATCAAGATCTTGAGGCATG |
| | | | ACATTGGTGCCACTGTCCATGAGCTTTCACGAGATAAGAAGAA |
| | | | AGACACAGTGCCCTGGTTCCCAAGAACCATTCAAGAGCTGGAC |
| | | | AGATTTGCCAATCAGATTCTCAGCTATGGAGCGGAACTGGATG |
| | | | CTGACCACCCTGGTTTTAAAGATCCTGTGTACCGTGCAAGACG |
| | | | GAAGCAGTTTGCTGACATTGCCTACAACTACCGCCATGGGCAG |
| | | | CCCATCCCTCGAGTGGAATACATGGAGGAAGAAAAGAAAACAT |
| | | | GGGGCACAGTGTTCAAGACTCTGAAGTCCTTGTATAAAACCCA |
| | | | TGCTTGCTATGAGTACAATCACATTTTTCCACTTCTTGAAAAG |
| | | | TACTGTGGCTTCCATGAAGATAACATTCCCCAGCTGGAAGACG |
| | | | TTTCTCAGTTCCTGCAGACTTGCACTGGTTTCCGCCTCCGACC |
| | | | TGTGGCTGGCCTGCTTTCCTCTCGGGATTTCTTGGGTGGCCTG |
| | | | GCCTTCCGAGTCTTCCACTGCACACAGTACATCAGACATGGAT |
| | | | CCAAGCCCATGTATACCCCCGAACCTGACATCTGCCATGAGCT |
| | | | GTTGGGACATGTGCCCTTGTTTTCAGATCGCAGCTTTGCCCAG |
| | | | TTTTCCCAGGAAATTGGCCTTGCCTCTCTGGGTGCACCTGATG |
| | | | AATACATTGAAAAGCTCGCCACAATTTACTGGTTTACTGTGGA |
| | | | GTTTGGGCTCTGCAAACAAGGAGACTCCATAAAGGCATATGGT |
| | | | GCTGGGCTCCTGTCATCCTTTGGTGAATTACAGTACTGCTTAT |
| | | | CAGAGAAGCCAAAGCTTCTCCCCCTGGAGCTGGAGAAGACAGC |
| | | | CATCCAAAATTACACTGTCACGGAGTTCCAGCCCCTCTATTAC |
| | | | GTGGCAGAGAGTTTTAATGATGCCAAGGAGAAAGTAAGGAACT |
| | | | TTGCTGCCACAATACCTCGGCCCTTCTCAGTTCGCTACGACCC |
| | | | ATACACCCAAAGGATTGAGGTCTTGGACAATACCCAGCAGCTT |
| | | | AAGATTTTGGCTGATTCCATTAACAGTGAAATTGGAATCCTTT |
| | | | GCAGTGCCCTCCAGAAAATAAAGTAA |
| WPREmut6 | 2433-3024 | 6 | AATCAACCTCTGGATTACAAAATTTGTGAAAGATTGACTGGTA |
| J04514 | | | TTCTTAACTATGTTGCTCCTTTTACGCTATGTGGATACGCTGC |
| | | | TTTAATGCCTTTGTATCATGCTATTGCTTCCCGTATGGCTTTC |
| | | | ATTTTCTCCTCCTTGTATAAATCCTGGTTGCTGTCTCTTTATG |
| | | | AGGAGTIGTGGCCCGTTGTCAGGCAACGTGGCGTGGTGTGCAC |
| | | | TGTGTTTGCTGACGCAACCCCCACTGGTTGGGGCATTGCCACC |
| | | | ACCTGTCAGCTCCTTTCCGGGACTTTCGCTTTCCCCCTCCCTA |
| | | | TTGCCACGGCGGAACTCATCGCCGCCTGCCTTGCCCGCTGCTG |
| | | | GACAGGGGCTCGGCTGTTGGGCACTGACAATTCCGTGGTGTTG |
| | | | TCGGGGAA [symbol] CGTCCTTTCC [symbol] TGGCTGCTCGCCTGTGTTG |
| | | | CCACCTGGATTCTGCGCGGGACGTCCTTCTGCTACGTCCCTTC |
| | | | GGCCCTCAATCCAGCGGACCTTCCTTCCCGCGGCCTGCTGCCG |
| | | | GCTCTGCGGCCTCTTCCGCGTCTTCGCCTTCGCCCTCAGACGA |
| | | | GTCGGATCTCCCTTTGGGCCGCCTCCCCGCCTG |
| | | | (the six point mutations - mutations of the |
| | | | putative promoter and the ATG start codon |
| | | | of the WHV-X open reading frame - are shown |
| | | | in boxes ) |
| bGH polyA | 3031-3255 | 7 | CTGTGCCTTCTAGTTGCCAGCCATCTGTTGTTTGCCCCTCCCC |
| signal | | | CGTGCCTTCCTTGACCCTGGAAGGTGCCACTCCCACTGTCCTT |
| | | | TCCTAATAAAATGAGGAAATTGCATCGCATTGTCTGAGTAGGT |
| | | | GTCATTCTATTCTGGGGGGTGGGGTGGGGCAGGACAGCAAGGG |
| | | | GGAGGATTGGGAAGACAATAGCAGGCATGCTGGGGATGCGGTG |
| | | | GGCTCTATGG |
| 3' ITR | 3283-3390 | 8 | AGGAACCCCTAGTGATGGAGTTGGCCACTCCCTCTCTGCGCGC |
| | | | TCGCTCGCTCACTGAGGCCGCCCGGGCTTTGCCCGGGCGGCCT |
| | | | CAGTGAGCGAGCGAGCGCGCAG |
| Human PAH | n/a | 9 | MSTAVLENPG LGRKLSDFGQ ETSYIEDNCN QNGAISLIFS |
| protein | | | LKEEVGALAK VLRLFEENDV NLTHIESRPS RLKKDEYEFF |
| | | | THLDKRSLPA LTNIIKILRH DIGATVHELS RDKKKDTVPW |
| | | | FPRTIQELDR FANQILSYGA ELDADHPGFK DPVYRARRKQ |
| | | | FADIAYNYRH GQPIPRVEYM EEEKKTWGTV FKTLKSLYKT |
| | | | HACYEYNHIF PLLEKYCGFH EDNIPQLEDV SQFLQTCTGF |
| | | | RLRPVAGLLS SRDFLGGLAF RVFHCTQYIR HGSKPMYTPE |
| | | | PDICHELLGH VPLFSDRSFA QFSQEIGLAS LGAPDEYIEK |
| | | | LATIYWFTVE FGLCKQGDSI KAYGAGLLSS FGELQYCLSE |
| | | | KPKLLPLELE KTAIQNYTVT EFQPLYYVAE SENDAKEKVR |
| | | | NFAATIPRPF SVRYDPYTQR IEVLDNTQQL KILADSINSE |
| | | | IGILCSALQK IK |

Complete Vector 1 sequence
CTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCTAGTAG
GCTCAGAGGCACACAGGAGTTTCTGGGCTCACCCTGCCCCCTTCCAACCCCTCAGTTCCCATCCTCCAGCA
GCTGTTTGTGTGCTGCCTCTGAAGTCCACACTGAACAAACTTCAGCCTACTCATGTCCCTAAAATGGGCAA
ACATTGCAAGCAGCAAACAGCAAACACACAGCCCTCCCTGCCTGCTGACCTTGGAGCTGGGGCAGAGGTCA
GAGACCTCTCTGGGCCCATGCCACCTCCAACATCCACTCGACCCCTTGGAATTTCGGTGGAGAGGAGCAGA
GGTTGTCCTGGCGTGGTTTAGGTAGTGTGAGAGGGGTACCCGGGGATCTTGCTACCAGTGGAACAGCCACT
AAGGATTCTGCAGTGAGAGCAGAGGGCCAGCTAAGTGGTACTCTCCCAGAGACTGTCTGACTCACGCCACC
CCCTCCACCTTGGACACAGGACGCTGTGGTTTCTGAGCCAGGTACAATGACTCCTTTCGGTAAGTGCAGTG
GAAGCTGTACACTGCCCAGGCAAAGCGTCCGGGCAGCGTAGGCGGGCGACTCAGATCCCAGCCAGTGGACT
TAGCCCCTGTTTGCTCCTCCGATAACTGGGGTGACCTTGGTTAATATTCACCAGCAGCCTCCCCCGTTGCC
CCTCTGGATCCACTGCTTAAATACGGACGAGGACAGGGCCCTGTCTCCTCAGCTTCAGGCACCACCACTGA
CCTGGGACAGTCAGGTAAGTATCAAGGTTACAAGACAGGTTTAAGGAGACCAATAGAAACTGGGCTTGTCG
AGACAGAGAAGACTCTTGCGTTTCTGATAGGCACCTATTGGTCTTACTGACATCCACTTTGCCTTTCTCTC
CACAGGCAATTGATCCCCCTGATCTGCGGCCTCGACGGTATCGATAAGCTTGCCACCATGTCCACTGCGGT
CCTGGAAAACCCAGGCTTGGGCAGGAAACTCTCTGACTTTGGACAGGAAACAAGCTATATTGAAGACAACT
GCAATCAAAATGGTGCCATATCACTGATCTTCTCACTCAAAGAAGAAGTTGGTGCATTGGCCAAAGTATTG
CGCTTATTTGAGGAGAATGATGTAAACCTGACCCACATTGAATCTAGACCTTCTCGTTTAAAGAAAGATGA
GTATGAATTTTTCACCCATTTGGATAAACGTAGCCTGCCTGCTCTGACAAACATCATCAAGATCTTGAGGC
ATGACATTGGTGCCACTGTCCATGAGCTTTCACGAGATAAGAAGAAAGACACAGTGCCCTGGTTCCCAAGA
ACCATTCAAGAGCTGGACAGATTTGCCAATCAGATTCTCAGCTATGGAGCGGAACTGGATGCTGACCACCC
TGGTTTTAAAGATCCTGTGTACCGTGCAAGACGGAAGCAGTTTGCTGACATTGCCTACAACTACCGCCATG
GGCAGCCCATCCCTCGAGTGGAATACATGGAGGAAGAAAAGAAAACATGGGGCACAGTGTTCAAGACTCTG
AAGTCCTTGTATAAAACCCATGCTTGCTATGAGTACAATCACATTTTTCCACTTCTTGAAAAGTACTGTGG
CTTCCATGAAGATAACATTCCCCAGCTGGAAGACGTTTCTCAGTTCCTGCAGACTTGCACTGGTTTCCGCC
TCCGACCTGTGGCTGGCCTGCTTTCCTCTCGGGATTTCTTGGGTGGCCTGGCCTTCCGAGTCTTCCACTGC
ACACAGTACATCAGACATGGATCCAAGCCCATGTATACCCCCGAACCTGACATCTGCCATGAGCTGTTGGG
ACATGTGCCCTTGTTTTCAGATCGCAGCTTTGCCCAGTTTTCCCAGGAAATTGGCCTTGCCTCTCTGGGTG
CACCTGATGAATACATTGAAAAGCTCGCCACAATTTACTGGTTTACTGTGGAGTTTGGGCTCTGCAAACAA
GGAGACTCCATAAAGGCATATGGTGCTGGGCTCCTGTCATCCTTTGGTGAATTACAGTACTGCTTATCAGA
GAAGCCAAAGCTTCTCCCCCTGGAGCTGGAGAAGACAGCCATCCAAAATTACACTGTCACGGAGTTCCAGC
CCCTCTATTACGTGGCAGAGAGTTTTAATGATGCCAAGGAGAAAGTAAGGAACTTTGCTGCCACAATACCT
CGGCCCTTCTCAGTTCGCTACGACCCATACACCCAAAGGATTGAGGTCTTGGACAATACCCAGCAGCTTAA
GATTTTGGCTGATTCCATTAACAGTGAAATTGGAATCCTTTGCAGTGCCCTCCAGAAAATAAAGTAATCTA
GAGGATCTCGAGAGATCTAATCAACCTCTGGATTACAAAATTTGTGAAAGATTGACTGGTATTCTTAACTA
TGTTGCTCCTTTTACGCTATGTGGATACGCTGCTTTAATGCCTTTGTATCATGCTATTGCTTCCCGTATGG
CTTTCATTTTCTCCTCCTTGTATAAATCCTGGTTGCTGTCTCTTTATGAGGAGTTGTGGCCCGTTGTCAGG
CAACGTGGCGTGGTGTGCACTGTGTTTGCTGACGCAACCCCCACTGGTTGGGGCATTGCCACCACCTGTCA
GCTCCTTTCCGGGACTTTCGCTTTCCCCCTCCCTATTGCCACGGCGGAACTCATCGCCGCCTGCCTTGCCC
GCTGCTGGACAGGGGCTCGGCTGTTGGGCACTGACAATTCCGTGGTGTTGTCGGGGAAATCATCGTCCTTT
CCTTGGCTGCTCGCCTGTGTTGCCACCTGGATTCTGCGCGGGACGTCCTTCTGCTACGTCCCTTCGGCCCT
CAATCCAGCGGACCTTCCTTCCCGCGGCCTGCTGCCGGCTCTGCGGCCTCTTCCGCGTCTTCGCCTTCGCC
CTCAGACGAGTCGGATCTCCCTTTGGGCCGCCTCCCCGCCTGGGATCTCTGTGCCTTCTAGTTGCCAGCCA
TCTGTTGTTTGCCCCTCCCCCGTGCCTTCCTTGACCCTGGAAGGTGCCACTCCCACTGTCCTTTCCTAATA
AAATGAGGAAATTGCATCGCATTGTCTGAGTAGGTGTCATTCTATTCTGGGGGGTGGGGTGGGGCAGGACA
GCAAGGGGGAGGATTGGGAAGACAATAGCAGGCATGCTGGGGATGCGGTGGGCTCTATGGACCGGTCTCGA
GATCCACTAGGGCCGCAGGAACCCCTAGTGATGGAGTTGGCCACTCCCTCTCTGCGCGCTCGCTCGCTCAC
TGAGGCCGCCCGGGCTTTGCCCGGGCGGCCTCAGTGAGCGAGCGAGCGCGCAG (SEQ ID NO: 10)

1. A nucleic acid construct comprising an expression cassette for human phenylalanine hydroxylase (PAH), wherein the expression cassette comprises a PAH-coding sequence linked operably to a mammalian promoter, the expression cassette further comprising a first RNA transcript enhancing element and a second RNA transcript enhancing element.

2. The construct of claim 1, wherein the construct further comprises an enhancer, optionally wherein the enhancer is a human apolipoprotein E (ApoE) enhancer or comprises SEQ ID NO: 2.

3. The construct of claim 1 or 2, wherein the construct comprises a promoter that is active in human liver cells, optionally wherein the liver cells are hepatocytes, optionally wherein the promoter is a human alpha-1 antitrypsin (AAT) promoter or comprises SEQ ID NO: 3.

4. The construct of any one of claims 1-3, wherein the first RNA transcript enhancing element comprises an intron, optionally wherein the intron is an HBB-IGG intron or comprises SEQ ID NO: 4.

5. The construct of any one of claims 1-4, wherein the PAH-coding sequence encodes a human PAH protein, optionally wherein:
the human PAH protein comprises SEQ ID NO: 9 or an amino acid sequence that is at least 95% identical to SEQ ID NO: 9, or
the PAH-coding sequence comprises SEQ ID NO: 5 or a codon-optimized variant of SEQ ID NO: 5.

6. The construct of any one of claims 1-5, wherein the second RNA transcript enhancing element comprises a WPRE element, optionally wherein the WPRE element is mutated from wildtype WPRE or comprises SEQ ID NO: 6.

7. The construct of any one of claims 1-6, wherein the first and second RNA transcript enhancing elements are located upstream and downstream, respectively, of the PAH-coding sequence, optionally wherein the first and second RNA transcript enhancing elements flank the PAH-coding sequence.

8. The construct of any one of claims 1-7, wherein the expression cassette comprises a polyA signal, optionally wherein the polyA signal is a bovine growth hormone polyA signal or comprises SEQ ID NO: 7.

9. The construct of claim 8, wherein the expression cassette comprises, from 5′ to 3′, an enhancer, the promoter, the first RNA transcript enhancing element, the PAH-coding sequence, the second RNA transcript enhancing element, and the polyA signal.

10. The construct of claim 1, wherein the construct comprises (i) from 5′ to 3′, SEQ ID NOs: 2, 3, 4, 5, 6, and 7; or (ii) SEQ ID NO: 10.

11. The construct of any one of claims 1-10, wherein the construct is a viral construct.

12. The construct of claim 11, wherein the construct is an adeno-associated viral construct, optionally wherein:
the construct comprises AAV inverted terminal repeats (ITR), optionally wherein the AAV ITRs are AAV2 ITRs, or
the construct comprises a 5′ ITR and a 3′ ITR comprising SEQ ID NOs: 1 and 8, respectively.

13. A recombinant adeno-associated virus (rAAV), comprising the construct of claim 12.

14. The rAAV of claim 13, wherein the rAAV:
(i) has a natural or chimeric AAV serotype,
(ii) is of serotype AAV6, AAV7, AAV8, or AAV9,
(iii) comprises capsid proteins derived from AAV2, AAV6, AAV7, AAV8, and/or AAV9,
(iv) comprises hybrid capsid proteins derived from more than one AAV serotype, optionally from AAV2, AAV6, and AAV9; and/or
(v) is hepatotropic.

15. The rAAV of claim 14, wherein the rAAV is rAAV6.

16. The rAAV of claim 14, wherein the rAAV is rAAV9.

17. The rAAV of any one of claims 13-16, wherein the rAAV is produced in insect cells, optionally wherein the insect cells are Sf9 cells.

18. The rAAV of any one of claims 13-16, wherein the rAAV is produced in mammalian cells, optionally wherein the mammalian cells are HEK293 cells.

19. A cell for producing the rAAV of any one of claims 13-18, wherein the cell comprises a nucleotide sequence coding for capsid proteins VP1, VP2, and VP3 of the rAAV, and the construct of claim 12.

20. The cell of claim 19, wherein the cell is an insect cell, optionally wherein the insect cell is an Sf9 cell.

21. The cell of claim 19, wherein the cell is a mammalian cell, optionally wherein the mammalian cell is an HEK293 cell.

22. A method of producing the rAAV of any one of claims 13-18, comprising
culturing the cell of any one of claims 19-21 under conditions that allow packaging of the rAAV, and
isolating the packaged rAAV.

23. A pharmaceutical composition comprising the construct of any one of claims 1-12 or the rAAV of any one of claims 13-18, and a pharmaceutically acceptable excipient.

24. A method of expressing human phenylalanine hydroxylase (PAH) protein in a mammalian cell, the method comprising introducing to the cell the construct of any one of claims 1-12, the rAAV of any one of claims 13-18, or the composition of claim 23.

25. The method of claim 24, wherein the mammalian cell is a human cell, optionally wherein the human cell is in or from a human in need of reduction of plasma phenylalanine level.

26. The method of claim 25, wherein the mammalian cell is a liver cell, optionally wherein the liver cell is a hepatocyte.

27. A human cell engineered by the method of any one of claims 24-26.

28. A method of treating phenylketonuria (PKU) in a human patient, the method comprising administering to the patient a therapeutically effective amount of the construct of any one of claims 1-12, the rAAV of any one of claims 13-18, the composition of claim 23, or the cell of claim 27.

29. A method of treating phenylketonuria (PKU) in a human patient, the method comprising administering to the patient a therapeutically effective amount of a nucleic acid construct comprising an expression cassette for human phenylalanine hydroxylase (PAH), wherein the construct is delivered by an rAAV that is rAAV9 or rAAV2/9.

30. The method of claim 29, wherein the construct is according to any one of claims 1-12.

31. The method of claim 29 or 30, wherein the rAAV9 or rAAV2/9 is produced in insect cells, optionally wherein the insect cells are Sf9 cells.

32. The method of claim 29 or 30, wherein the rAAV9 or rAAV2/9 is produced in mammalian cells, optionally wherein the mammalian cells are HEK293 cells.

33. The method of any one of claims 29-32, wherein the method comprises administering to the patient a therapeutically effective amount of the rAAV9 or rAAV2/9.

34. The method of any one of claims 29-32, wherein the method comprises administering to the patient a therapeutically effective amount of a pharmaceutical composition comprising the construct and a pharmaceutically acceptable excipient.

35. The method of any one of claims 29-32, wherein the method comprises administering to the patient a therapeutically effective amount of a mammalian cell comprising the construct.

36. The method of claim 35, wherein the mammalian cell is a liver cell, optionally wherein the liver cell is a hepatocyte.

37. The method of any one of claims 28-36, wherein the PKU is classic, moderate, mild, or variant PKU.

38. The method of any one of claims 28-37, comprising administering to the patient the rAAV intravenously or via direct injection.

39. The method of any one of claims 28-38, wherein the treatment results in (i) a decrease in phenylalanine (Phe) levels by at least 30% or by at least 90%, or (ii) Phe levels of less than 360 μmol/L, in a blood sample from the patient.

40. Use of the construct of any one of claims 1-12, the rAAV of any one of claims 13-18, the composition of claim 23, or the cell of claim 27, for the manufacture of a medicament for treating phenylketonuria according to the method of any one of claims 28 and 37-39.

41. Use of a nucleic acid construct comprising an expression cassette for human phenylalanine hydroxylase (PAH), wherein the construct is delivered by an rAAV9 or rAAV2/9, for the manufacture of a medicament for treating phenylketonuria in a human patient according to the method of any one of claims 29-39.

42. The construct of any one of claims 1-12, the rAAV of any one of claims 13-18, the composition of claim 23, or the cell of claim 27, for use in treating phenylketonuria according to the method of any one of claims 28 and 37-39.

43. A nucleic acid construct comprising an expression cassette for human phenylalanine hydroxylase (PAH), wherein the construct is delivered by an rAAV9 or rAAV2/9, for use in treating phenylketonuria in a human patient according to the method of any one of claims 29-39.


SEQUENCE LISTING (10 sequences)

SEQ ID NO: 1 - DNA, 130 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 321 bp, Homo sapiens
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 3 - DNA, 393 bp, Homo sapiens
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 4 - DNA, 133 bp, Artificial Sequence
  source: /note=“Description of Artificial Sequence Synthetic polynucleotide”
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 5 - DNA, 1359 bp, Unknown
  source: /note=“Description of Unknown PAH cDNA sequence”
atgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcagttcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctctattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaa

SEQ ID NO: 6 - DNA, 592 bp, Artificial Sequence
  source: /note=“Description of Artificial Sequence Synthetic polynucleotide”
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 7 - DNA, 225 bp, Artificial Sequence
  source: /note=“Description of Artificial Sequence Synthetic polynucleotide”
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 8 - DNA, 108 bp, Artificial Sequence
  source: /note=“Description of Artificial Sequence Synthetic polynucleotide”
aggaacccctagtgatggagttggccactccctctctgcgcgctcgctcgctcactgaggccgcccgggctttgcccgggcggcctcagtgagcgagcgagcgcgcag

SEQ ID NO: 9 - protein, 452 aa, Homo sapiens
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 10 - DNA, 3390 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggcaattgatccccctgatctgcggcctcgacggtatcgataagcttgccaccatgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcagttcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctctattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaatctagaggatctcgagagatctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgggatctctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtctcgagatccactagggccgcaggaacccctagtgatggagttggccactccctctctgcgcgctcgctcgctcactgaggccgcccgggctttgcccgggcggcctcagtgagcgagcgagcgcgcag